SEQUENCE LISTING (14 sequences)

SEQ ID NO: 1 - DNA, 1474 bp, Marine actinomycete
gtttgattccctgctcaggacgaacgctggcggcgtgcttaacacatgcaagtcgaacgatgatccggtttcggccggtgattagtggcgaacgggtgagtaacacgtgggtaatctgccctgcactctgggataagcccgggaaactgggtctaataccggatatgaccttcggtcgcatgatcgttggtggaaagcttttgcggtgtgggatgggcccgcggcctatcagcttgttggtggggtgatggcctaccaaggcgacgacgggtagccggcctgagagggtgaccggccacactgggactgagacacggcccagactcctacgggaggcagcagtggggaatattgcacaatgggcgcaagcctgatgcagcgacgccgcgtgagggatgacggccttcgggttgtaaacctctttcagcagggaagaagcgcaagtgacggtacctgcagaagaagcaccggctaactacgtgccagcagccgcggtaatacgtagggtgcaagcgttgtccggaattattgggcgtaaagagctcgtaggcggcttgtcgcgtcggttgtgaaagcccggggcttaaccctgggtctgcagtcgatacgggcaggctagagttcggtaggggagactggaattcctggtgtagcggtgaaatgcgcagatatcaggaggaacaccggtggcgaaggcgggtctctgggccgatactgacgctgaggagcgaaagcgtggggagcgaacaggattagataccctggtagtccacgctgtaaacggtgggaactaggtgtgggcagcattccacgttgtctgtgccgtagctaacgcattaagttccccgcctggggagtacggccgcaaggctaaaactcaaaggaattgacgggggcccgcacaagcggcggagcatgtggcttaattcgacgcaacgcgaagaaccttaccaaggcttgacatgcatcggaagcatccagagatgggtgtgctctttgagtcggtgtacaggtggtgcatggctgtcgtcagctcgtgtcgtgagatgttgggttaagtcccgcaacgagcgcaacccttgtcctgtgttgccagcggagccttcgggctgccggggactcacgggagactgccggggtcaactcggaggaaggtggggacgacgtcaagtcatcatgccccttatgtcttgggctgcacacgtgctacaatggccggtacaatgagctgcgatgccgtgaggtggagcgaatctcaaaaagccggtctcagttcggattggggtctgcaactcgaccccatgaagtcggagtcgctagtaatcgcagatcagcattgctgcggtgaatacgttcccgggccttgtacacaccgcccgtcacgtcacgaaagtcggtaacacccgaagccggtggcctaaccccttgtgggagggagtcgtcgaaggtgggactggcgattgggacgaagtcg

SEQ ID NO: 2 - DNA, 1492 bp, Marine actinomycete
agagtttgatcctggctcaggacgaacgctggcggcgtgcttaacacatgcaagtcgaacgatgatccggtttcggccggtgattagtggcgaacgggtgagtaacacgtgggtaatctgccctgcactctgggataagcccgggaaactgggtctaataccggatatgaccttcggtcgcatgatcgttggtggaaagcttttgcggtgtgggatgggcccgcggcctatcagcttgttggtggggtgatggcctaccaaggcgacgacgggtagccggcctgagagggtgaccggccacactgggactgagacacggcccagactcctacgggaggcagcagtggggaatattgcacaatgggcgcaagcctgatgcagcgacgccgcgtgagggatgacggccttcgggttgtaaacctctttcagcagggaagaagcgcaagtgacggtacctgcagaagaagcaccggctaactacgtgccagcagccgcggtaatacgtagggtgcaagcgttgtccggaattattgggcgtaaagagctcgtaggcggcttgtcgcgtcggttgtgaaagcccggggcttaaccctgggtctgcagtcgatacgggcaggctagagttcggtaggggagactggaattcctggtgtagcggtgaaatgcgcagatatcaggaggaacaccggtggcgaaggcgggtctctgggccgatactgacgctgaggagcgaaagcgtggggagcgaacaggattagataccctggtagtccacgctgtaaacggtgggaactaggtgtgggcagcattccacgttgtctgtgccgtagctaacgcattaagttccccgcctggggagtacggccgcaaggctaaaactcaaaggaattgacgggggcccgcacaagcggcggagcatgtggcttaattcgacgcaacgcgaagaaccttaccaaggcttgacatgcatcggaagcatccagagatgggtgtgctctttgagtcggtgtacaggtggtgcatggctgtcgtcagctcgtgtcgtgagatgttgggttaagtcccgcaacgagcgcaacccttgtcctgtgttgccagcggagccttcgggctgccggggactcacgggagactgccggggtcaactcggaggaaggtggggacgacgtcaagtcatcatgccccttatgtcttgggctgcacacgtgctacaatggccggtacaatgagctgcgatgccgtgaggtggagcgaatctcaaaaagccggtctcagttcggattggggtctgcaactcgaccccatgaagtcggagtcgctagtaatcgcagatcagcattgctgcggtgaatacgttcccgggccttgtacacaccgcccgtcacgtcacgaaagtcggtaacacccgaagccggtggcctaaccccttgtgggagggagtcgtcgaaggtgggactggcgattgggacgaagtcgtaacaaggtagccgta

SEQ ID NO: 3 - DNA, 1492 bp, Marine actinomycete
agagtttgatcctggctcaggacgaacgctggcggcgtgcttaacacatgcaagtcgaacgatgatccggtttcggccggtgattagtggcgaacgggtgagtaacacgtgggtaatctgccctgcactctgggataagcctgggaaactgggtctaataccggatatgaccttcggtcgcatgatcgttggtggaaagcttttgcggtgtgggatgggcccgcggcctatcagcttgttggtggggtgatggcctaccaaggcgacgacgggtagccggcctgagagggtgaccggccacactgggactgagacacggcccagactcctacgggaggcagcagtggggaatattgcacaatgggcgcaagcctgatgcagcgacgccgcgtgagggatgacggccttcgggttgtaaacctctttcagcagggaagaagcgcaagtgacggtacctgcagaagaagcaccggctaactacgtgccagcagccgcggtaatacgtagggtgcaagcgttgtccggaattattgggcgtaaagagctcgtaggcggcttgtcgcgtcggttgtgaaagcccggggcttaaccctgggtctgcagtcgatacgggcaggctagagttcggtaggggagactggaattcctggtgtagcggtgaaatgcgcagatatcaggaggaacaccggtggcgaaggcgggtctctgggccgatactgacgctgaggagcgaaagcgtggggagcgaacaggattagataccctggtagtccacgctgtaaacggtgggaactaggtgtgggcagcattccacgttgtctgtgccgtagctaacgcattaagttccccgcctggggagtacggccgcaaggctaaaactcaaaggaattgacgggggcccgcacaagcggcggagcatgtggcttaattcgacgcaacgcgaagaaccttaccaaggcttgacatgcatcggaagcatccagagatgggtgtgctctttgagtcggtgtacaggtggtgcatggctgtcgtcagctcgtgtcgtgagatgttgggttaagtcccgcaacgagcgcaacccttgtcctgtgttgccagcggagctttcgggctgccggggactcacgggagactgccggggtcaactcggaggaaggtggggacgacgtcaagtcatcatgccccttatgtcttgggctgcacacgtgctacaatggccggtacaatgagctgcgatgccgtgaggtggagcgaatctcaaaaagccggtctcagttcggattggggtctgcaactcgaccccatgaagtcggagtcgctagtaatcgcagatcagcattgctgcggtgaatacgttcccgggccttgtacacaccgcccgtcacgtcacgaaagtcggtaacacccgaagccggtggcctaaccccttgtgggagggagtcgtcgaaggtgggactggcgattgggacgaagtcgtaacaaggtagccgta

SEQ ID NO: 4 - DNA, 1492 bp, Marine actinomycete
agagtttgatcctggctcaggacgaacgctggcggcgtgcttaacacatgcaagtcgaacgatgatccggtttcggccggtgattagtggcgaacgggtgagtaacacgtgggtaatctgccctgcactctgggataagcctgggaaactgggtctaataccggatatgaccttcggtcgcatgatcgttggtggaaagcttttgcggtgtgggatgggcccgcggcctatcagcttgttggtggggtagtggcctaccaaggcgacgacgggtagccggcctgagagggtgaccggccacactgggactgagacacggcccagactcctacgggaggcagcagtggggaatattgcacaatgggcgaaagcctgatgcagcgacgccgcgtgagggatgacggccttcgggttgtaaacctctttcagcagggaagaagcgcaagtgacggtacctgcagaagaagcaccggctaactacgtgccagcagccgcggtaatacgtagggtgcaagcgttgtccggaattattgggcgtaaagagctcgtaggcggcttgtcgcgtcggttgtgaaagcccggggcttaaccctgggtctgcagtcgatacgggcaggctagagttcggtaggggagactggaattcctggtgtagcggtgaaatgcgcagatatcaggaggaacaccggtggcgaaggcgggtctctgggccgatactgacgctgaggagcgaaagcgtggggagcgaacaggattagataccctggtagtccacgctgtaaacggtgggaactaggtgtgggcagcattccacgttgtctgtgccgcagctaacgcattaagttccccgcctggggagtacggccgcaaggctaaaactcaaaggaattgacgggggcccgcacaagcggcggagcatgtggcttaattcgacgcaacgcgaagaaccttaccaaggcttgacatgcatcggaagcgcctagagatgggtgtgctctttgagtcggtgtacaggtggtgcatggctgtcgtcagctcgtgtcgtgagatgttgggttaagtcccgcaacgagcgcaacccttgtcctgtgttgccagcggagccttcgggctgccggggactcacgggagactgccggggtcaactcggaggaaggtggggacgacgtcaagtcatcatgccccttatgtcttgggctgcacacgtgctacaatggccggtacaatgagctgcgatgccgtgaggtggagcgaatctcaaaaagccagtctcagttcggattggggtctgcaactcgaccccatgaagtcggagtcgctagtaatcgcagatcagcattgctgcggtgaatacgttcccgggccttgtacacaccgcccgtcacgtcacgaaagtcggtaacacccgaagccggtggcctaaccccttgtgggagggagtcgtcgaaggtgggactggcgattgggacgaagtcgtaacaaggtagccgta

SEQ ID NO: 5 - DNA, 1503 bp, Marine actinomycete
agagtttgatcctggctcaggacgaacgctggcggcgtgcttaacacatgcaagtcgaacgatgatccggcttcggtcggggattagtggcgaacgggtgagtaacacgtgggcaatctgccctgcactctgggataagcctgggaaactgggtctaataccggatatgaccttctctcgcatgggggagggtggaaagcttttgcggtgcaggatgggcccgcggcctatcagcttgttggtggggtagtggcctaccaaggcgacgacgggtagccggcctgagagggtgaccggccacactgggactgagacacggcccagactcctacgggaggcagcagtggggaatattgcacaatgggcgcaagcctgatgcagcgacgccgcgtgagggatgacggccttcgggttgtaaacctctttcagcagggaagaagccttgcctttttgggtgggtgacggtacttgcagaagaagcaccggctaactacgtgccagcagccgcggtaatacgtagggtgcaagcgttgtccggatttattgggcgtaaagagctcgtaggcggcttgtcgcgtcgattgtgaaagctcagggcttaaccctgggtctgcagtcgatacgggcaggctagagttcggtaggggagactggaattcctggtgtagcggtgaaatgcgcagatatcaggaggaacaccggtggcgaaggcgggtctctgggccgatactgacgctgaggagcgaaagcgtggggagcgaacaggattagataccctggtagtccacgctgtaaacggtgggaactaggtgtgggcagcattccacgttgtctgtgccgtagctaacgcattaagttccccgcctggggagtacggccgcaaggctaaaactcaaaggaattgacgggggcccgcacaagcggcggagcatgtggcttaattcgacgcaacgcgaagaaccttaccaaggcttgacatacgccggaaaaccatggagacatggtccctctttgagtcggtgtacaggtggtgcatggctgtcgtcagctcgtgtcgtgagatgttgggttaagtcccgcaacgagcgcaacccttatcctgtgttgccagcaactctcttcggaggggttggggactcacgggagactgccggggtcaactcggaggaaggtggggacgacgtcaagtcatcatgccccttatgtcttgggctgcacacgtgctacaatggctggtacaatgagctgcgatgccgtgaggtggagcgaatctcaaaaagccagtctcagttcggattggggtctgcaactcgaccccatgaagtcggagtcgctagtaatcgcagatcagcattgctgcggtgaatacgttcccgggccttgtacacaccgcccgtcacgtcacgaaagtcggtaacacccgaagccggtggcctaacccccttgtggggagggagtcgtcgaaggtgggactggcgattgggacgaagtcgtaacaaggta

SEQ ID NO: 6 - DNA, 1508 bp, Marine actinomycete
agagtttgatccctggctcaggacgaacgctggcggcgtgcttaacacatgcaagtcgaacgatgatccggtttcggccggggattagtggcgaacgggtgagtaacacgtgggtaatctgccctgcactttgggataagcctgggaaactgggtctaataccggatatgaccttccttcgcatgggggttggtggaaagcttttgcggtgcaggatggacccgcggcctatcagcttgttggtggggtagtggcctaccaaggcgacgacgggtagccggcctgagagggtgaccggccacactgggactgagacacggcccagactcctacgggaggcagcagtggggaatattgcacaatgggcgaaagcctgatgcagcgacgccgcgtgagggatgacggccttcgggttgtaaacctctttcagcagggaagaagccttgcctttttgggtgggtgacggtacttgcagaagaagcaccggctaactacgtgccagcagccgcggtaatacgtagggtgcaagcgttgtccggatttattgggcgtaaagagctcgtaggcggcttgtcacgtcgattgtgaaagctcagggcttaaccctgggtctgcagtcgatacgggcaggctagagttcggtaggggagactggaattcctggtgtagcggtgaaatgcgcagatatcaggaggaacaccggtggcgaaggcgggtctctgggccgatactgacgctgaggagcgaaagcgtggggagcgaacaggattagataccctggtagtccacgctgtaaacggtgggaactaggtgtgggcagcattccacgttgtctgtgccgcagctaacgcattaagttccccgcctggggagtacggccgcaaggctaaaactcaaaggaattgacgggggcccgcacaagcggcggagcatgtggcttaattcgacgcaacgcgaagaaccttaccaaggcttgacatacatcggaatctgctggagacagtagcgctctttgagtcggtgtacaggtggtgcatggctgtcgtcagctcgtgtcgtgagatgttgggttaagtcccgcaacgagcgcaacccttattctgtgttgccagcatgccctttcgggggtgatggggactcacgggagactgccggggtcaactcggaggaaggtggggacgacgtcaagtcatcatgccccttatgtcttgggctgcacacgtgctacaatggctggtacaatgagctgcgataccgtgaggtggagcgaatctcaaaaagccagtctcagttcggattggggtctgcaactcgaccccatgaagtcggagtcgctagtaatcgcagatcagcattgctgcggtgaatacgttcccgggccttgtacacaccgcccgtcacgtcacgaaagtcggtaacacccgaagccggtggcctaaccccttgtgggagggagctgtcgaaggtgggactggcgattgggacgaagtcgtaacaaggtagccgta

SEQ ID NO: 7 - DNA, 1445 bp, Marine actinomycete
agagtttgatccctggctcaggacgaacgctggcggcgtgcttaacacatgcaagtcgaacgatgaaccggtttcggccggggattagtggcgaacgggtgagtaacacgtgggtgacctgccctgcactctgggataagcccgggaaactgggtctaatactggatatgaccggtggccgcatggtctgccggtggaaagctttatgcggtgtgggatgggcccgcggcctatcagcttgttggtggggtgatggcctaccaaggcgacgacgggtagccggcctgagagggtgaccggccacactgggactgagacacggcccagactcctacgggaggcagcagtggggaatattgcacaatgggcggaagcctgatgcagcgacgccgcgtgagggatgacggccttcgggttgtaaacctctttcagcagggaagaagcgcaagtgacggtacctgcagaagaagcaccggctaactacgtgccagcagccgcggtaatacgtagggtgcaagcgttgtccggaattattgggcgtaaagagctcgtaggcggcctgtcgcgtcgattgtgaaagcccggggcttaactccgggtctgcagtcgatacgggcaggctagagttcggtaggggagactggaattcctggtgtagcggtgaaatgcgcagatatcaggaggaacaccggtggcgaaggcgggtctctgggccgatactgacgctgaggagcgaaagcgtggggagcgaacaggattagataccctggtagtccacgctgtaaacggtgggaactaggtgtgggcgacattccacgttgtctgtgccgtagctaacgcattaagttccccgcctggggagtacggccgcaaggctaaaactcaaaggaattgacgggggcccgcacaagcggcggagcatgtggcttaattcgacgcaacgcgaagaaccttaccaaggcttgacatacgccggaaatctctggagacaggggctcccttttgggccggtgtacaggtggtgcatggctgtcgtcagctcgtgtcgtgagatgttgggttaagtcccgcaacgagcgcaacccttgtcctgtgttgccagcaacaccttcgggtggttggggactcacgggagactgccggggtcaactcggaggaaggtggggacgacgtcaagtcatcatgccccttatgtcttgggctgcacacgtgctacaatggccggtacaaagggctgcgatgccgtgaggcggagcgaatcccaaaaagccggtctcagttcggattggggtctgcaactcgaccccatgaagtcggagtcgctagtaatcgcagatcagcagtgctgcggtgaatacgttcccgggccttgtacacaccgcccgtcacgtcacgaaagtcggtaacacccgaagccggggcctaaccctctgggagggagccgt

SEQ ID NO: 8 - RNA, 1541 nt, Escherichia coli
aaauugaagaguuugaucauggcucagauugaacgcuggcggcaggccuaacacaugcaagucgaacgguaacaggaaacagcuugcuguuucgcugacgaguggcggacgggugaguaaugucugggaaacugccugauggagggggauaacuacuggaaacgguagcuaauaccgcauaacgucgcaagaccaaagagggggaccuucgggccucuugccaucagaugugcccagaugggauuagcuaguaggugggguaacggcucaccuaggcgacgaucccuagcuggucugagaggaugaccagccacacuggaacugagacacgguccagacuccuacgggaggcagcaguggggaauauugcacaaugggcgcaagccugaugcagccaugccgcguguaugaagaaggccuucggguuguaaaguacuuucagcggggaggaagggaguaaaguuaauaccuuugcucauugacguuacccgcagaagaagcaccggcuaacuccgugccagcagccgcgguaauacggagggugcaagcguuaaucggaauuacugggcguaaagcgcacgcaggcgguuuguuaagucagaugugaaauccccgggcucaaccugggaacugcaucugauacuggcaagcuugagucucguagagggggguagaauuccagguguagcggugaaaugcguagagaucuggaggaauaccgguggcgaaggcggcccccuggacgaagacugacgcucaggugcgaaagcguggggagcaaacaggauuagauacccugguaguccacgccguaaacgaugucgacuuggagguugugcccuugaggcguggcuuccggagcuaacgcguuaagucgaccgccuggggaguacggccgcaaguuaaaacucaaaugaauugacgggggcccgcacaagcgguggagcaugugguuuaauucgaugcaacgcgaagaaccuuaccuggucuugacauccacggaaguuuucagagaugagaaugugccuucgggaaccgugagacaggugcugcauggcugucgucagcucguguugugaaauguuggguuaagucccgcaacgagcgcaacccuuauccuuuguugccagcgguccggccgggaacucaaaggagacugccagugauaaacuggaggaagguggggaugacgucaagucaucauggcccuuacgaccagggcuacacacgugcuacaauggcgcauacaaagagaagcgaccucgcgagagcaagcggaccucauaaagugcgucguaguccggauuggagucugcaacucgacuccaugaagucggaaucgcuaguaaucguggaucagaaugccacggugaauacguucccgggccuuguacacaccgcccgucacaccaugggaguggguugcaaaagaaguagguagcuuaaccuucgggagggcgcuuaccacuuugugauucaugacuggggugaagucguaacaagguaaccguaggggaaccugcgguuggaucaccuccuua

SEQ ID NO: 9 - DNA, 20 bp, Artificial sequence
  Amplification primer
agagtttgatcctggctcag

SEQ ID NO: 10 - DNA, 22 bp, Artificial sequence
  Amplification primer
tacggctaccttgttacgactt

SEQ ID NO: 11 - DNA, 19 bp, Artificial sequence
  Amplification primer
gtgccagcagccgcggtaa

SEQ ID NO: 12 - DNA, 16 bp, Artificial sequence
  Amplification primer
gcaacgagcgcaaccc

SEQ ID NO: 13 - DNA, 19 bp, Artificial sequence
  Amplification primer
ccgcggctgctggcacgta

SEQ ID NO: 14 - DNA, 19 bp, Artificial sequence
  Amplification primer
gtgcgggcccccgtcaatt

(19) United States
(12) Patent Application Publication
(10) Pub. No.: US 2005/0153389 A1
(43) Pub. Date: Jul. 14, 2005

(54) Method for the production of bioactive substances from the novel actinomycete taxon MAR2 ("Marinophilus")

(75) Inventors: William H. Fenical, Del Mar, CA (US); Paul R. Jensen, San Diego, CA (US); Tracy J. Mincer, Cambridge, MA (US)

Correspondence Address: DLA PIPER RUDNICK GRAY CARY US, LLP, 4365 EXECUTIVE DRIVE, SUITE 1100, SAN DIEGO, CA, 92121-2133, US

(73) Assignee: The University of California, Oakland, CA (US)

(21) Appl. No.: 10/972,530

(22) Filed: Oct. 25, 2004

Related U.S. Application Data
(60) Provisional application No. 60/514,127, filed on Oct. 24, 2003.

(51) Int. Cl. C12P 1/00; C12P 19/62; C12N 1/20

(52) U.S. Cl. 435/41; 435/252.35

(57) ABSTRACT

The present invention provides methods for the isolation of a new group of actinomycetes and for the identification of members of this group based on characteristic nucleotide sequences and phylogenetic analyses. This group is phylogenetically distinct from all other known actinomycetes and represents a novel genus that includes multiple new species. Members of this group can be recognized by signature MAR2 nucleotides present in their 16S rRNA gene sequences and by standard phylogenetic treeing methods.

### RELATED APPLICATIONS

This application claims the benefit under 35 U.S.C. § 119(e) of U.S. provisional patent application 60/514,127, filed Oct. 24, 2003, and under 35 U.S.C. § 120 of U.S. patent application Ser. No. 10/873,657, filed Jun. 21, 2004, which are incorporated herein by reference in their entirety.

This invention was made with government support Grant No. CA 44848 awarded by National Institute of Health. The United States government has certain rights in this invention.

### BACKGROUND OF THE INVENTION

1. Field of the Invention

The invention relates generally to methods for the isolation of a micoorganism and the use of the microorganism to produce biologically active agents, and more specifically to a genus of actinomycetes and methods for producing biomolecules using the genus.

2. Background Information

Pharmaceutical researchers have long tapped actinomycetes, gram-positive, soil bacteria with fungal-like filaments, as a source of novel antibiotics and antitumor biomolecules. In addition, pharmaceutical researchers have long been using natural antibiotics from actinomycetes as models and starting materials for the production of new medicines. Prominent examples are the antibiotics actinomycin, streptomycin, and vancomycin. The repertoire of these microorganisms, previously known to live mainly in soil, has by now been well studied. Approximately 120 drugs have their origins in terrestrial actinomycetes, and although highly prolific for more than 50 years, the chemical diversity from this source began to decline more than 10 years ago.

Marine actinomycetes have only recently been discovered. Like their terrestrial relatives, these organisms make some highly biologically active substances. A U.S. team has recently discovered a new antitumor agent in one of these marine bacteria. The biomolecule salinosporamide A is a potent inhibitor of several types of human cancers. This biomolecule is produced by marine actinomycete strain CNB-392, a member of a new bacterial genus called “Salinospora.” Salinospora strains have been recovered from muddy sediments collected in shallow, as well as sediments collected in excess of 1,000 meters from the Atlantic and Pacific Oceans, the Red Sea, and the Gulf of California. In the deep oceans, there is no light, very high pressure, and low temperature.

New methods have also been developed for sifting through the samples (which contain roughly one billion microorganisms per cubic centimeter), identifying specific microorganisms by genetic methods and screening their metabolic products for anticancer and antibiotic properties. From 100 strains of Salinospora preliminarily tested, 80% produced molecules that inhibit cancer cell growth and roughly 35% revealed the ability to kill pathogenic bacteria and fungi. Salinosporamide A is a powerful inhibitor of certain colon and lung and breast cancers, and it has been shown to act by the inhibition of the intracellular proteasome.

However, the culture of marine organisms has proven difficult in the laboratory. A number of explanations have been proposed to explain this phenomenon. From an eco-physiological point of view, it has been argued that there exist obligate oligotrophs that cannot grow in any nutrient rich medium, such as is commonly used to grow land bacteria. It has even been proposed that sudden exposure to nutrient rich external conditions induces suicide responses originating from an imbalance between anabolism and catabolism. Thus there is a need in the art for improved methods for isolating and culturing marine bacteria, such as actinomycetes.

Antibiotic resistance of pathogenic bacteria, including pathogenic actinobacteria, such as Mycobacterium tuberculosis, is a well-known problem faced by medical practitioners in treatment of bacterial diseases. Therefore, there is a further need in the art for new antibiotics and drugs effective to circumvent resistance to existing antibiotics in treatment of bacterial infections in humans and in other mammals, including domestic and farm animals.

Many types of cancer cells also exhibit drug resistance. Accordingly, there is also an urgent need for new anticancer agents, for example, those with new pharmacological properties and unusual structures that may circumvent resistance to existing antibiotics.

### SUMMARY OF THE INVENTION

The present invention provides a new marine actinomycete taxon labeled MAR2, for which the genus name “Marinophilus” is proposed. Evidence is presented herein, that this new taxon represents a significant source of biologically active agents.

Accordingly, in one embodiment, the present invention provides an isolated marine actinomycete being a member of a new genus comprising a uridine at position 304 of a 16S RNA gene, a cytidine at position 671 of the 16S rRNA gene, a guanidine at position 735 of the 16S rRNA gene, as numbered by reference to the E. coli strain MG1655 sequence alignment of FIG. 3. Members of this group form a distinct clade using standard phylogenetic treeing methods and all members of this taxon are by definition more closely related to MAR2 clade members than to organisms that fall outside of the lade.

In another embodiment, the invention provides methods for producing a biomolecule having an activity of interest by culturing an invention marine actinomycete of the MAR2 group in a salt-containing growth medium to allow production of at least one biomolecule. The marine actinomycete or the growth medium containing the at least one biomolecule is collected and the biomolecule is extracted from the marine actinomycete cells or from the growth medium. The extracted biomolecule is tested for the presence of the activity of interest to produce a biomolecule having the activity of interest.

In still another embodiment, the invention provides methods for drug discovery wherein the method includes growing a strain of an invention actinomycete of the MAR2 group (“Marinophilus”) in salt-containing growth medium, collecting the actinomycete or the growth medium, and analyzing the actinomycete or the growth medium for the presence of a biomolecule with pharmacological activity.

In yet another embodiment, the invention provides methods for producing a biomolecule by growing an invention marine actinomycete of the MAR2 group in a salt-containing growth medium to produce the biomolecule; collecting the marine actinomycete or the growth medium containing the biomolecule; and extracting the biomolecule from the marine actinomycete or the growth medium to produce the biomolecule.

### BRIEF DESCRIPTION OF THE DRAWINGS

FIG. 1 illustrates phylogenetic relationships determined from nearly full 16s rDNA sequences of select MAR2 isolates and representatives of the two currently accepted genera within the Streptomycetaceae, Kitasatospora (abbreviated K.) and Streptomyces (abbreviated S.) (Anderson and Wellington, 2001; Zhang, et al. 1997. The phylogram was generated using a neighbor-joining method. Bootstrap values were calculated from 1000 re-samplings and are shown at their respective nodes if a value of 60% or greater was calculated. Glycomyces tenuis, Pilimelia anulata, and Micromonospora olivasterospora were used as outgroups.

FIGS. 2A-G provide DNA sequences encoding 16S rRNA gene sequences for MAR2 isolates. FIG. 2A=CNQ695 (SEQ ID NO: 1); FIG. 2B=CNQ03 (SEQ ID NO:2); FIG. 2C=CNQ732 (SEQ ID NO:3); FIG. 2D=CNR252 (SEQ ID NO:4); FIG. 2E=CNPO₂₇ (SEQ ID NO:5); FIG. 2F=CNQ140 (SEQ ID NO:6); and FIG. 2G=CNQ259 (SEQ ID NO:7).

FIGS. 3A-3B provide nucleotides 4033120 to 4034661 of E. coli strain MG 1655 rrsA gene sequence (SEQ ID NO:8) used for definition of the signature nucleotides in the 16S rRNA gene of MAR2 isolates. This sequence is part of GenBank entry: U00096 (Blattner et al., Science, 277 (5331), p. 1453-1474 (1997)). The dashes are inserted as positioning spacers to align the sequence to a standard secondary structure template (or “molecular ruler”) developed by the Ribosomal Database Project hosted by Michigan State University (Cole et al., (2003)) for alignment of prokaryote sequences. A dash in this alignment sequence does not represent a base, but rather is a positioner used to align the E. coli sequence to the standard secondary structure template for comparison.

### DETAILED DESCRIPTION OF THE INVENTION

This invention describes a method to access a new taxonomic group of marine actinomycetes for industrial purposes. Members of this new taxon produce biologically active metabolites and have the potential to produce new classes of biomolecules with entirely new mechanisms of action. Thus, accessing this group may lead to the discovery of new pharmaceutical agents that are superior to those available today. This discovery is significant as these microorganisms are new to science and represent a resource of untold magnitude.

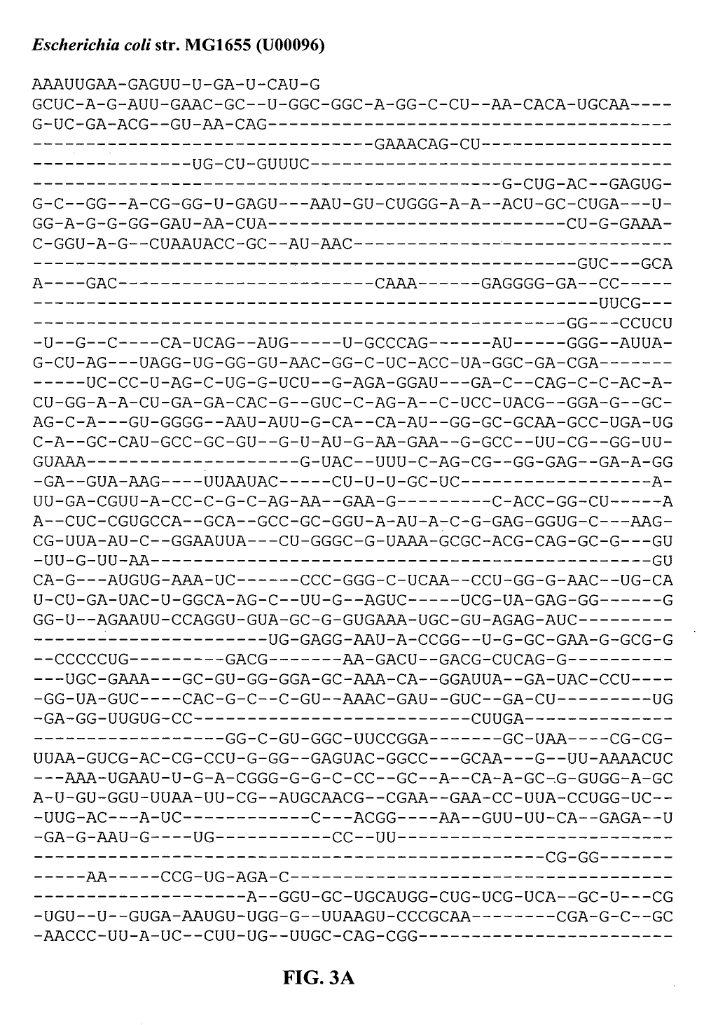

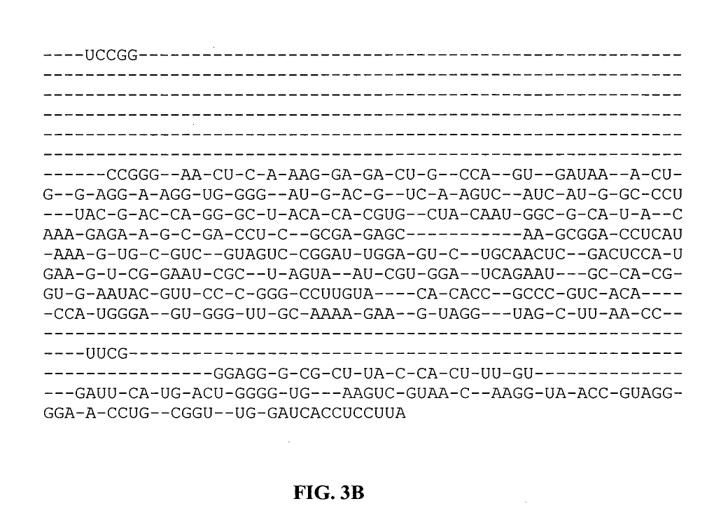

In one embodiment, novel strains of marine actinomycetes of the MAR2 group (“Marinophilus”) are described. The newly isolated marine actinomycete bacteria belong to a new taxon that can be recognized by 16S rRNA gene sequence analyses. By definition, members of this group form a coherent phylogenetic clade, using standard treeing methods such as PAUP 4.0 (Sinauer Associates, Inc., Sunderland, Mass.), and all members of this clade are more closely related to each other than to strains that fall outside of the clade. In addition, MAR2 members can be recognized by characteristic signature nucleotides as follows: a uridine at position 304 thereof, a cytidine at position 671 thereof, and a guanidine at position 735 thereof. It is possible that some MAR2 strains will not have all of these signatures and that some strains with these signatures will not belong to the MAR2 group. Accordingly, in one embodiment, the present invention provides an isolated marine actinomycete being a member of a new taxon comprising a uridine at position 304 of a 16S RNA gene, a cytidine at position 671 of the 16S rRNA gene, a guanidine at position 735 of the 16S rRNA, as numbered by reference to the alignment of E. coli strain MG1655 of FIG. 3.

In one embodiment, isolated MAR2 actinomycetes are characterized as having defined 16S rRNA genes. Such rRNA genes may be transcribed from a nucleotide sequence that includes the DNA sequence as shown in SEQ ID NO: 1, SEQ ID NO: 2, SEQ ID NO: 3, SEQ ID NO: 4, SEQ ID NO: 5, SEQ ID NO: 6 or SEQ ID NO: 7 as set forth in the Sequence Listing. In certain aspects, the isolated marine actinomycete comprises all family-specific signature nucleotides of the family Streptomycetaceae. Furthermore, in certain aspects the isolated marine actinomycete has a 16S rRNA sequence encoded by a nucleotide sequence that is 80%, 90%, 95%, or 99% identical to at least one of the nucleotide sequences of SEQ ID NOS: 1-7. The isolated marine actinomycete having the above-listed variant nucleotide sequences, further may be cultured in a sodium containing medium.

In a related aspect, on Jun. 20, 2003, strain CNQ140 was deposited under accession number PTA-5276 at the American Type Culture Collection (ATCC), Manassas, Va., USA, under the terms of the Budapest Treaty on the International Recognition of the Deposit of Microorganisms for the Purposes of Patent Procedure and Regulations thereunder (Budapest Treaty), and are thus maintained and made available according to the terms of the Budapest Treaty. Availability of such strains is not to be construed as a license to practice the invention in contravention of the rights granted under the authority of any government in accordance with its patent laws.

The isolated marine actinomycetes belonging to the MAR2 taxon disclosed herein were cultivated from samples collected from various locations near San Diego and the island of Guam.

The invention provides methods for producing a biomolecule having a therapeutic activity by growing the MAR2 marine actinomycetes in a salt-containing medium to produce a conditioned medium, and extracting a biomolecule with therapeutic activity or another desired biological activity from the marine actinomycete or the conditioned medium in which the MAR2 marine actinomycetes is grown.

The culturing can be performed, for example, in A1 medium. Extracting can be performed, for example, with the adsorbent resin Amberlite XAD-7, which in certain aspects, is eluted with acetone.

In another aspect, the invention MAR2 marine actinomycetes can be grown in the sodium-containing culture medium for 1 to 15 days, preferably 2 to 7 days, with or without shaking so as to obtain an in vitro culture of the invention MAR2 marine actinomycetes. The culture medium can be collected for extraction therefrom of excreted biomolecules of interest or the actinomycetes can be obtained from the culture for extraction of biomolecules of interest, such as at least one metabolite. In a related aspect, the metabolites may have anticancer, antifungal or antibiotic activity.

In another embodiment, bacterial preparations include one or more isolated and purified strain(s) of marine actinomycetes that fall within the MAR2 group are envisaged where the bioactive compositions containing metabolites are produced by cultivating the strains on medium/compositions as described above. Such organisms may include actinomycetes that fall within the MAR2 phylotype within the Streptomycetaceae based on 16S rRNA gene sequence analysis using, for example, methods of sequencing and tree construction such as PAUP (Phylogenetic Analysis Using Parsimony, Florida State University). MAR2 clade members will always group together in a phylogenetic tree and be more closely related to each other than to any other tree member.

In another embodiment, a method is disclosed for producing and isolating bioactive compositions having antibiotic and/or anticancer properties from one or more strains of actinomycetes, belonging to the MAR2 group. Strains belonging to this group may be cultured in a medium that may contain, but is not limited to, a sodium-containing culture medium. For example, the medium may include a certain amount of seawater or salts, various nutrients such as yeast extract, peptone, starch and glucose, and the like. Strains may be cultured in liquid medium, semi-solid, or on solid surfaces (e.g., agar).

Further, the resulting cultured strains may be extracted with an absorbent resin (e.g., but not limited to, Amberlite XAD-7) and subsequently eluted with an organic solvent, for example, a polar organic solvent such as acetone. The cultures may also be extracted with an organic solvent (e.g., ethyl acetate) or freeze-dried and extracted with an organic solvent (e.g., methanol). In a related aspect, the resulting solution is evaporated and the remaining solute is solubilized in a chaotropic agent (e.g., DMSO), where the solubilized residue contains the bioactive composition.

In another embodiment, a bioactive composition is isolated from extracts of cultured strains of marine actinomycetes belonging to the MAR2 group using, for example, column chromatography, HPLC, counter-current chromatography, or any methods familiar to one skilled in the art. Once in pure form, NMR and other spectral methods can resolve the structures of these biomolecules. As used herein, “extracts” means for example, whole cells, cell fragments, components, mixtures of uncharacterized molecules and compounds, and individual molecules and compounds.

In a related aspect, the secondary metabolites may be assayed for pharmaceutical, agrichemical, or other biotechnological related activities. For example, isolated bioactive compositions are effective against methylicillin-resistant Staphylococcus aureus (MRSA) and HCT-116 human carcinoma cells.

In one embodiment, isolated nucleic acids including SEQ ID NO: 1, SEQ ID NO: 2 SEQ ID NO: 3, SEQ ID NO: 4, SEQ ID NO: 5, SEQ ID NO: 6 or SEQ ID NO: 7 are envisaged, where such nucleic acids may be DNA or RNA. In a related aspect, such nucleic acids may include linkage to a vector. In a further aspect, a host cell may harbor the vector containing the nucleic acids as described above.

In another embodiment, a method for identifying strains that belong to the MAR2 clade is envisaged, including performing BLAST comparison searches of a public or private databases using, for example, default parameters, where 16S rRNA gene sequences of the bacterial strains are compared via a sequence comparison interface of the database. Further, text files can be generated which comprise a subset of resulting hits, where the text files are analyzed on a public or private ribosomal database using a sequence matching interface. Moreover, sequence homologies between the subset of hits from the BLAST search and sequences for MAR2 strains of bacteria can be determined and strains belonging to the MAR2 group based on the similarity between sequence alignments that are generated by the sequence matching interface of the ribosomal database can be identified.

The present invention also envisages methods for drug discovery which includes growing a strain of a marine actinomycete of the MAR2 group, collecting an actinomycete extract or conditioned growth medium and analyzing the extract or medium for the presence of biomolecules having pharmaceutical activity. The biomolecule, for example, may be a pharmaceutical agent, an antibiotic agent, an antifungal agent, and/or an anticancer agent. The genes responsible for the production of the molecule may be cloned and expressed in an heterologous host.

In a related aspect, bioactive compositions comprising such pharmaceutically active biomolecules and a pharmaceutically acceptable carrier are contemplated.

The following examples are intended to illustrate but not limit the invention.

### EXAMPLE 1

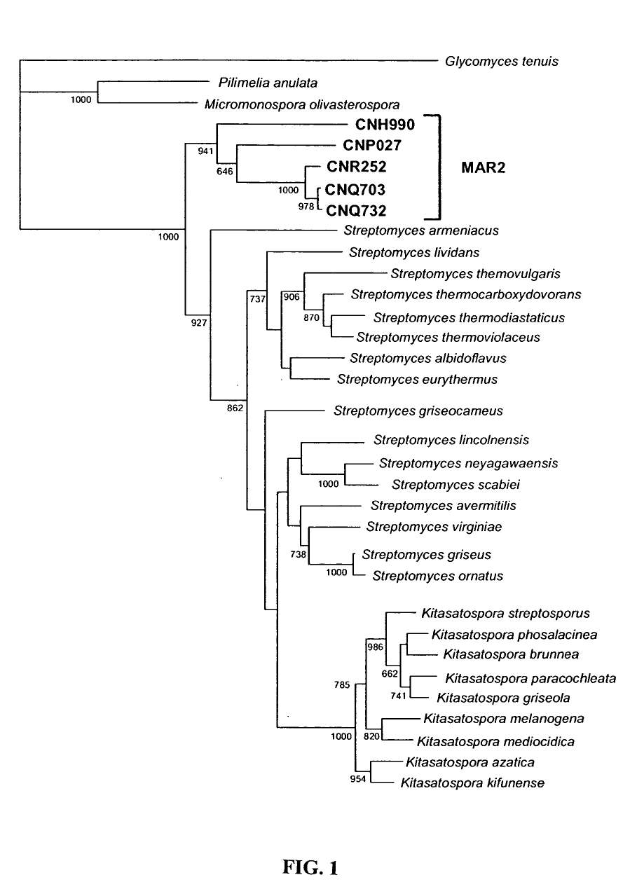

This invention provides methods for the isolation of a new group of actinomycetes and for the identification of members of this group based on characteristic nucleotide sequences. This group is phylogenetically distinct from all other known actinomycetes (FIG. 1) and represents a novel genus that includes multiple new species. Members of this group can be recognized by their characteristic nucleotide sequences. Certain strains can be cultured from marine sediments that have been air-dried, ground with a sterile mortar and pestle, and replicate stamped with a sterile sponge on A1 medium (1% starch, 0.4% yeast extract, 0.2% peptone, 1.6% agar, 100% seawater). For the production of biologically active substances, such strains are cultured in liquid A1 medium and the whole culture extracted with the adsorbent resin Amberlite XAD-7. The resin is eluted with acetone and the acetone removed by rotary evaporation. Extracts are solubilized in DMSO and tested for anticancer activities in laboratory assays. Thus far the following strains have been tested for activity against the human colon tumor cell line HCT-116 with the crude extracts demonstrating a range of cytotoxic activities: CNH990 <0.8 microgm/ml, CNQ703=21.0 microgm/ml, CNQ732=59.7 microgm/ml, CNR252 =44.4 micorgm/ml. Chromatographic separation of strain CNH990 extracted products has led to fractions with increased potency and complex NMR spectra indicating the presence of structurally-unprecedented bioactive metabolites.

The methods to isolate and identify the new actinomycete taxon described here provide organisms that are a source of new biologically active metabolites. These metabolites will have potential utility as new medicines and for other natural product applications.

Genomic DNA obtained from pure cultures of MAR 2 isolates was amplified using the PCR, employing the following general eubacterial primers:

| | |
|---|---|
| FC27 | |
| (5′-AGAGTTTGATCCTGGCTCAG-3′) | (SEQ ID NO:9) |
| and | |
| RC1492 | |
| (5′-TACGGCTACCTTGTTACGACTT-3′). | (SEQ ID NO:10) |

The PCR products were purified using a Qiagen QIAquick™ PCR cleanup kit following the manufacturers protocols (Qiagen Inc., Chatsworth, Calif.). Sequencing of both top and bottom strands of the entire PCR product was performed using forward primer, FC27, along with additional forward primers:
F514 (5′-GTGCCAGCAGCCGCGGTAA-3′) (SEQ ID NO:11)and
F1114 (5′-GCAACGAGCGCAACCC-3′) (SEQ ID NO:12)
and the reverse primer RC1492 along with additional reverse primers:
R530 (5′-CCGCGGCTGCTGGCACGTA-3′) (SEQ ID NO:13) and
   R936 (GTGCGGGCCCCCGTCAATT) (SEQ ID NO:14). Contiguous sequences were assembled and aligned to secondary 16S rRNA gene sequences.

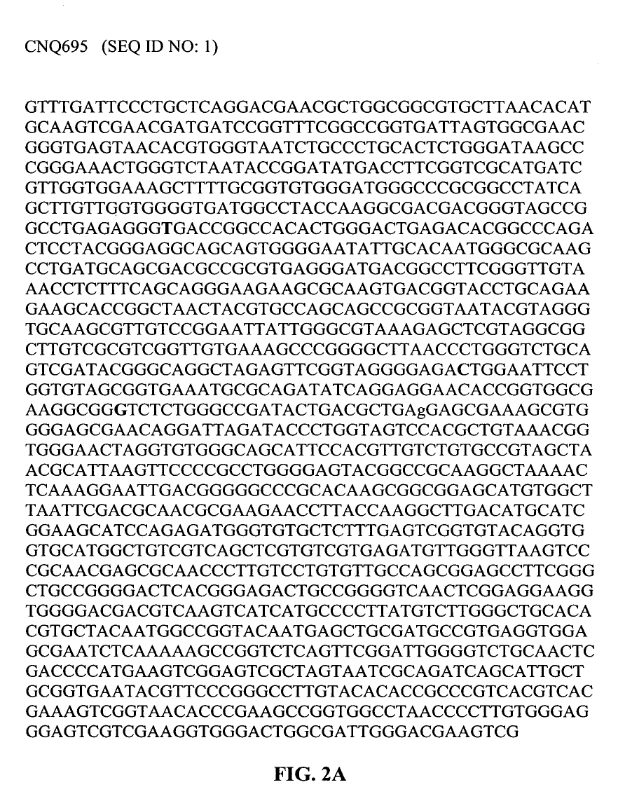

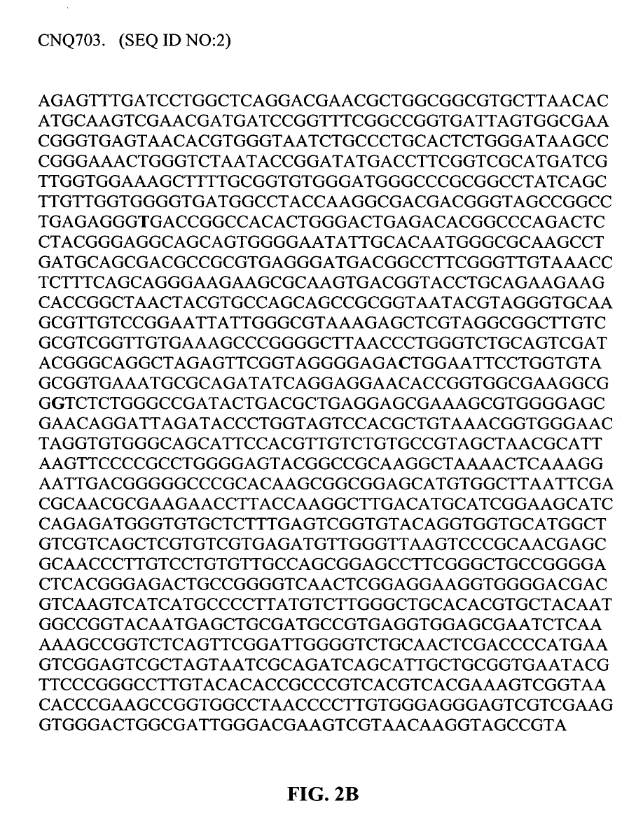

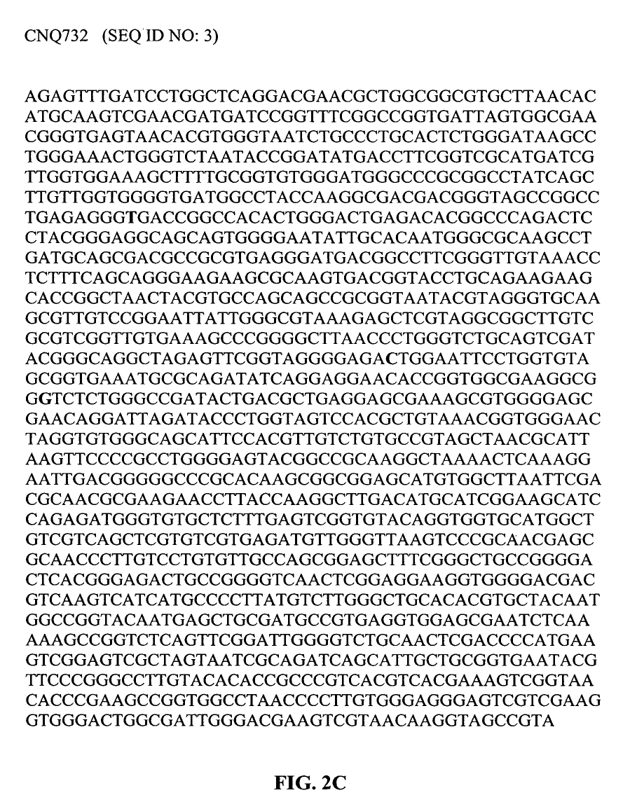

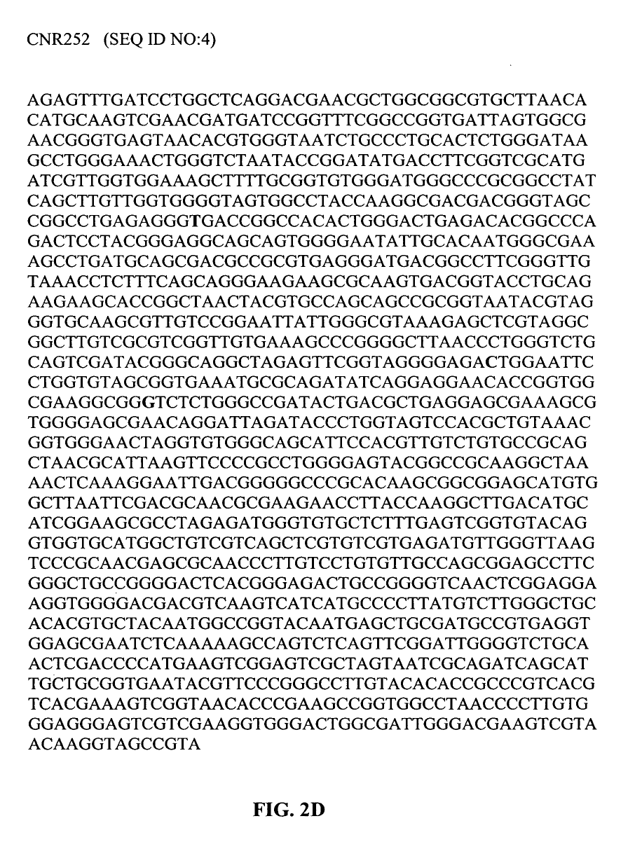

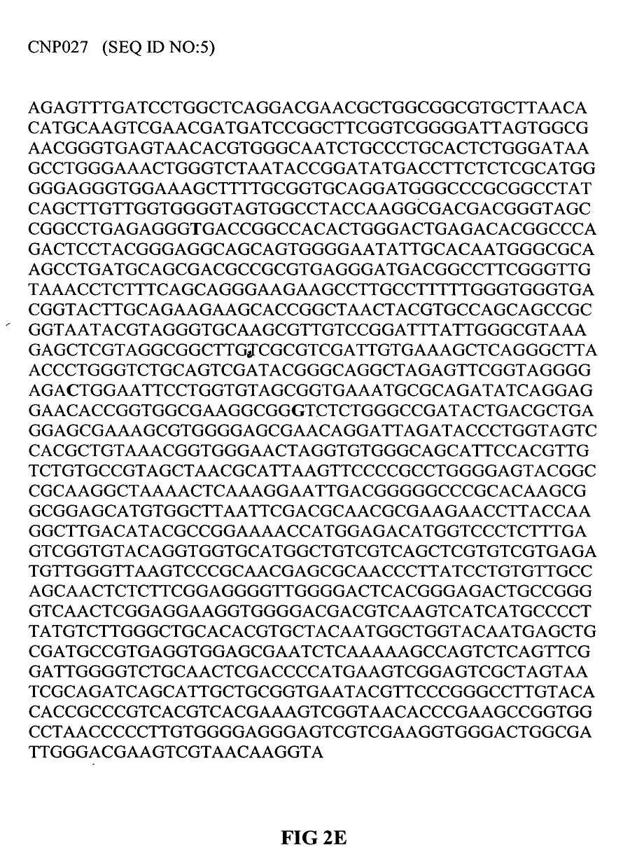

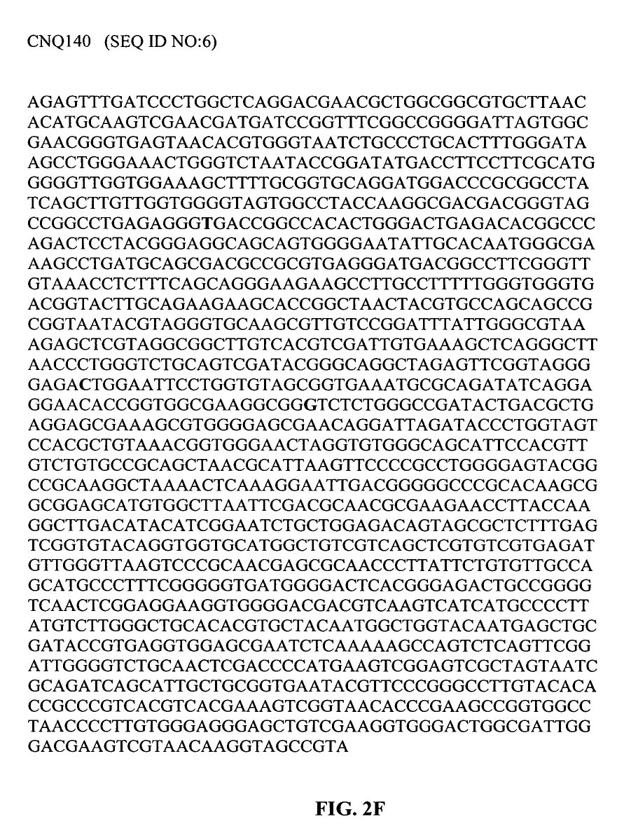

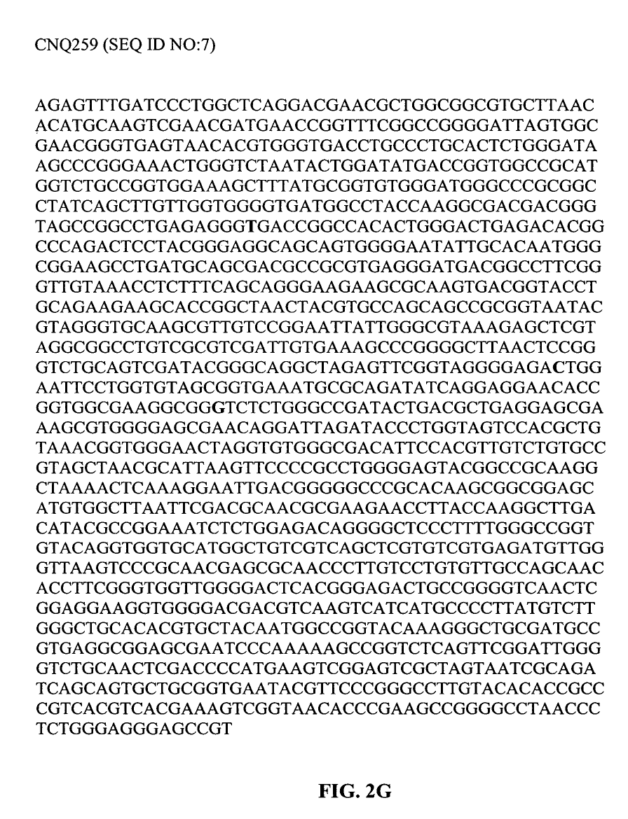

Alignments within the RDP (Ribosomal Database Project) version 8.1 (updated 12-19-O₂; B. L. Maidak et al. (2001) Nucleic Acid Res 29: 173-174) were used to compare sequences of 16S rRNA of all currently known MAR2 isolates to the secondary structure of the 16S rRNA gene (rrsA) sequence of Escherichia coli strain MG1655 (FIG. 3). The procedure of aligning to secondary structures obtained from a public database is a standard method to create a “molecular ruler” by which signature nucleotides are reported. Further procedures used for sequence alignment of 16S rRNA sequences using the RDP methods entitled “Ribosomal Database Project II” are available on the internet at rdp.cme.msu.edu. The 16S rRNA gene sequences (1486-1503 bp depending on strain) of all known MAR2 isolates (SEQ ID NOS:1-7) (See FIG. 2) were analyzed from nucleotide positions 8-1507 using this E. coli numbering system. The 16S rRNA signature nucleotides distinguishing the Marinomyces (MAR2) clade from all other genera within the family Streptmycetaceae, as determined by this procedure, are described above.

### EXAMPLE 2

For extraction of biomolecules from the strain CNQ140 MAR2 actinomycete, the following protocol was used. Pre-washed Amberlite XAD-7 resin (20 g) was added to 1 liter of culture and mixed for 1 hour. The contents were collected by filtration, washed with deionized water (1 liter), and then eluted with acetone (250 ml). The extraction liquid from each successive extraction step was tested for tumor cell cytotoxicity (IC₅₀) against HCT-116 colon cancer cell line. The first step of the extraction yielded an extract fraction, Q140-3, which showed an IC₅₀ of 1.2 microgm/ml against the colon cancer cell line HCT-116. A second purification step using C-18 reverse phase HPLC and a solvent mixture of 65% MeCN in water yielded four distinct biomolecules, all of which possess activity against the colon cancer cell line. These CNQ140-derived biomolecules kill or substantially inhibit growth of drug resistant pathogenic bacteria as well.

As shown by the data in Table 1 below, the biomolecules obtained by the invention methods from MAR 2 strain CNQ140 have both anticancer and antibiotic activity, as illustrated in tests against HCT-116 human colon adenocarcinoma cancer cells, methicillin resistant Staphylococcus aureus (MRSA), and vancomycin-resistant Enterococcus faecium (VREF):

TABLE I

| | | MRSA | VREF |
|---|---|---|---|
| | HCT-116 | (MIC | (MIC |
| Biomolecule | (IC₅₀ microgm/ml) | microgm/ml) | microgm/ml) |
| CNQ140.996a | 0.18 | 0.13 | 0.13 |
| CNQ140.996b | 2.9 | 0.25 | 0.63 |
| CNQ140.996c | 2.6 | 0.25 | 0.25 |
| CNQ140.1010 | 3.1 | 0.25 | 0.25 |

### Table of References

Mincer T J, Jensen P R, Kauffman C A, Fenical W. Widespread and persistent populations of a major new marine actinomycete taxon in ocean sediments. Appl. Environ. Microbiol. 68: 5005-5011 (incorporated herein in its entirety, by reference).
Maidak B L, Cole J R, Lilburn T G, Parker C T, Saxman P R, Farris R J, Garrity G M, Olsen G J, Schimidt T M, Tiedje J M (2001) The RDP-II (Ribosomal Database Project). (incorporated herein in its entirety, by reference).

Although embodiments of the invention have been described with reference to the above examples, it will be understood that modifications and variations also are encompassed.

1. An isolated marine bacterium wherein the marine actinomycete is a member of a new genus comprising a 16S rRNA gene with MAR2 characteristic signature nucleotides:
a uridine at position 304 thereof, a cytidine at position 671 thereof, and a guanidine at position 735.

2. The isolated marine actinomycete of claim 1, wherein the 16S rRNA gene is encoded by a nucleic acid comprising a sequence as set forth in SEQ ID NO: 1, 2, 3, 4, 5, 6 or 7.

3. The isolated marine actinomycete of claim 2, wherein the isolated marine actinomycete has a 16S rRNA gene sequence encoded by a nucleotide sequence that is about 80% to about 99% identical to at least one of the nucleotide sequences of SEQ ID NOS: 1-7).

4. The isolated marine actinomycete of claim 1, wherein the genus further comprises all family-specific signature nucleotides of the actinomycete family Streptomycetaceae.

5. A method for producing a biomolecule having an activity of interest, comprising:
culturing a marine actinomycete of claim 1 in a salt-containing growth medium to allow production of at least one biomolecule;
collecting the marine actinomycete or the growth medium containing the at least one biomolecule;
extracting the biomolecule from the marine actinomycete or the growth medium; and
testing the extracted biomolecule for the presence of the activity of interest to produce a biomolecule having the activity of interest.

6. The method of claim 5, wherein the activity of interest is pharmaceutical activity selected from antibiotic, antifungal, antiviral and anticancer activities.

7. The method of claim 6, wherein the pharmaceutical activity is an antibiotic activity.

8. The method of claim 6, wherein the pharmaceutical activity is an antifungal activity.

9. The method of claim 6, wherein the pharmaceutical activity is an anti-cancer activity.

10. The method of claim 5, wherein the growth medium comprises seawater.

11. A method for drug discovery comprising:
growing a strain of actinomycete of claim 1 in salt-containing growth medium;
collecting the actinomycete or the growth medium, and
analyzing the actinomycete or growth medium for the presence of a biomolecule with pharmacological activity.

12. The method of claim 11, wherein the analysis comprises an assay for antibacterial activity.

13. The method of claim 12, wherein the analysis comprises an assay for anti-viral activity.

14. The method of claim 12, wherein the analysis comprises an assay for anti-cancer activity.

15. The method of claim 17, wherein the analysis comprises an assay for anti-fungal activity.

16. The method of claim 17, wherein the growth medium comprises seawater.

17. A method for producing a biomolecule, comprising
growing a marine actinomycete of claim 1 in a salt-containing growth medium to produce the biomolecule;
collecting the marine actinomycete or the growth medium containing the biomolecule; and
extracting the biomolecule from the marine actinomycete or the growth medium to produce the biomolecule.

18. The method of claim 17, wherein the growth medium comprises seawater.

19. The method of claim 17, wherein the extracting comprises fractionating the growth medium.

20. The method of claim 19, wherein the extracting comprises use of an absorbent resin.

21. The method of claim 19, wherein the absorbent resin is eluted with an organic solvent.

22. The method of claim 16, wherein the biomolecule is a secondary metabolite of the marine actinomycete.
